(19) United States
(12) Patent Application Publication
(10) Pub. No.: US 2026/0196402 A1
(43) Pub. Date: Jul. 9, 2026

(54) FLUOROSCOPY-TRANSLUCENT HIGH-CURRENT ELECTROMAGNETIC FLAT TRANSMITTER

(71) Applicant: Magnisity Ltd., Hod-HaSharon (IL)

(72) Inventors: Benjamin GREENBURG, Hod-HaSharon (IL); Ron BARAK, Tel-Aviv (IL)

(73) Assignee: Magnisity Ltd., Hod-HaSharon (IL)

(21) Appl. No.: 19/133,267

(22) PCT Filed: Nov. 29, 2023
(86) PCT No.: PCT/IL2023/051224

§ 371 Date: May 28, 2025

Related U.S. Application Data
(60) Provisional application No. 63/428,584, filed on Nov. 29, 2022.

(51) Int. Cl. H01F 27/28

(52) U.S. Cl. CPC H01F 27/2804; H01F 2027/2809

(57) ABSTRACT

The present invention discloses systems comprising flat electromagnetic (EM) transmitters having a negligible interference with other devices, and methods of generating thereof.

### RELATED APPLICATION/S

This application claims the benefit of priority of U.S. Provisional Patent Application No. 63/428,584, filed on 29 Nov. 2022, the contents of which are incorporated herein by reference in their entirety.

### FIELD AND BACKGROUND OF THE INVENTION

The present invention, in some embodiments thereof, relates to flat electromagnetic (EM) transmitters and, more particularly, but not exclusively, to flat electromagnetic (EM) transmitters having a negligible interference with other devices.

In a traditional EM system, sensors are usually made of coils. They sense the transmitted fields due to Faraday's law of induction. Since the induced voltage on a coil is proportional to the transmitted frequency, it is therefore advantageous in these systems to use rather high frequencies (for example >1 KHz) to amplify the pickup on the sensor's coils. In this case, the amplitude of the transmitted fields need not be too high, since the pickup amplification is mainly achieved by increasing the transmitted frequencies. However, increasing the frequency also increases the eddy-currents induced in metals surrounding the system, for example, through metal bars in the patient's bed.

An EM transmitter may generate multiple different EM fields. For example, each EM field is modulated using a different frequency, for example in the range of 1-40 KHz. The different fields may be used for position and/or orientation tracking of an EM sensor, usually a coil-based sensor. The sensor may sense a superposition of the different EM fields, for example according to Faraday's law of induction. Then, the sensed signal may be decomposed, for example by a processor, into multiple amplitudes by using frequency decomposition methods such as Discrete Fourier Transform (DFT), correlation methods or any other suitable method.

Additional background art includes U.S. Pat. No. 6,833,814 disclosing a system and method for tracking the position and orientation of a probe such as a catheter whose transverse inner dimension may be at most about two millimeters. Three planar antennas that at least partly overlap are used to transmit electromagnetic radiation simultaneously, with the radiation transmitted by each antenna having its own spectrum. In the case of single-frequency spectra, the antennas are provided with mechanisms for decoupling them from each other. A receiver inside the probe includes sensors of the three components of the transmitted field, with sensors for at least two of the three components being pairs of sensors, such as coils, disposed symmetrically with respect to a common reference point. In one variant of the receiver, the coils are collinear and are wound about cores that are mounted in pairs of diametrically opposed apertures in the housing of the probe. In another variant of the receiver-catheter combination, the catheter is configured with an inner and outer sleeve connected at their ends by one or more flexible elements on which the coils are mounted. Each member of a pair of coils that sense the same component of the transmitted field is connected to a different input of a differential amplifier. The position and orientation of the receiver relative to the antennas are determined non-iteratively, by setting up an overdetermined set of linear equations that relates the received signals to transmitter-receiver amplitudes, solving for the amplitudes and inferring the position coordinates and the orientation angles of the receiver relative to the transmitter from these amplitudes.

U.S. Pat. No. 10,615,500 discloses a computer-implemented method of designing an antenna assembly for radiating an electromagnetic field for electromagnetic navigation. Multiple diagonal lines are computed, relative to a coordinate system of a substrate having a boundary, based on a seed rectangle having multiple vertices. Each diagonal line bisects a respective vertex of the seed rectangle, and extends from that vertex to the boundary. For each diagonal line, distances between adjacent pairs of planar antenna vertices to be positioned along the respective diagonal line are determined, and the planar antenna vertices are positioned along the respective diagonal line based on the determined distances. The distances increase in a direction from the respective vertex of the seed rectangle to the boundary. A planar antenna layout is generated by interconnecting the planar antenna vertices by way of respective straight linear portions to form multiple loops that sequentially traverse each of the diagonal lines.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. An electromagnetic (EM) transmitter, comprising a plurality of EM transmitting coils positioned one on top another; each of said plurality of EM transmitting coils having at least one conductive trace; said EM transmitter defining a flat surface;
wherein a calculated quantity of conductive trace material in a section along an axis perpendicular to said flat surface of said EM transmitter is homogenous or quasi-homogenous when compared with all other sections in said EM transmitter.

Example 2. The EM transmitter according to example 1, wherein said plurality of EM transmitting coils are positioned with an offset in relation to one another in order to generate said homogenous or quasi-homogenous quantity of conductive trace material.

Example 3. The EM transmitter according to example 1 or example 2, further comprising at least one additional layer; said additional layer comprising one or more regions; said regions having one or more of at least one conductive trace or copper configured for providing material required to achieve said homogenous or quasi-homogenous quantity of conductive material.

Example 4. The EM transmitter according to any one of examples 1-3, wherein one or more EM transmitting coil from said plurality of EM transmitting coils further comprise one or more regions; said regions having one or more of at least one conductive trace or copper configured for providing material required to achieve said homogenous or quasi-homogenous quantity of conductive material.

Example 5. The EM transmitter according to any one of examples 1-4, wherein said plurality of EM transmitting coils are configured to be used with low frequencies and high electrical currents with smaller power dissipation.

Example 6. The EM transmitter according to any one of examples 1-5, wherein:
a. said low frequencies are lower than 1 kHz; and
b. said high electrical currents above 0.3 Ampere or above 1 Ampere.

Example 7. The EM transmitter according to any one of examples 1-6, wherein said plurality of EM transmitting coils are configured to be used with a combination of:
a. low frequencies and high electrical currents; and
b. high frequencies and low electrical currents.

Example 8. The EM transmitter according to any one of examples 1-7, wherein:
a. said low frequencies are lower than 1 kHz;
b. said high electrical currents above 0.3 Ampere or above 1 Ampere;
c. said high frequencies are from 1 kHz to 40 kHz; and
d. said low currents are below 1 Ampere.

Example 9. The EM transmitter according to any one of examples 1-8, wherein said conductive trace material is one or more of copper, silver or any other compatible material.

Example 10. The EM transmitter according to any one of examples 1-9, wherein spacings between parts of said at least one trace are from about 1 mil to about 5 mil.

Example 11. The EM transmitter according to any one of examples 1-10, wherein spacings between parts of said at least one trace are larger than 5 mil.

Example 12. The EM transmitter according to any one of examples 1-11, wherein said at least one conductive trace comprises a weight of from about 0.5 oz to about 20 oz.

Example 13. The EM transmitter according to any one of examples 1-12, each EM transmitting coil from said plurality of EM transmitting coils comprise a geometry of said at least one conductive trace.

Example 14. The EM transmitter according to any one of examples 1-13, wherein said geometry is one or more of square, rectangular, triangular, diagonal, circular, or any other geometrical form.

Example 15. The EM transmitter according to any one of examples 1-14, wherein each EM transmitting coil from said plurality of EM transmitting coils is made of a plurality of sub-transmitting coils.

Example 16. The EM transmitter according to any one of examples 1-15, further comprising an isolating layer between each sub-transmitting coil from said plurality of sub-transmitting coils.

Example 17. The EM transmitter according to any one of examples 1-16, wherein said offset is characterized by a direction and a size.

Example 18. The EM transmitter according to any one of examples 1-17, wherein said direction is one or more of up, down, left and right.

Example 19. The EM transmitter according to any one of examples 1-18, wherein said direction is one or more of in the X axis and/or in the Y axis.

Example 20. The EM transmitter according to any one of examples 1-19, wherein said size is from about 0.01 mm to about 10 mm.

Example 21. The EM transmitter according to any one of examples 1-20, wherein said at least one additional layer is an invert layer or contains copper regions which serve as invert copper to other layers.

Example 22. A tracking system comprising:
a. an EM transmitter according to example 1,
b. a fluoroscope.

Example 23. A method for generating a homogenous or quasi-homogenous distribution of copper over an area of an EM transmitter by offset positioning, comprising:
a. identifying a location of traces and spacing on a plurality of EM coil layers;
b. positioning a first EM coil layer in a fixed position thereby generating a reference layer for said offset;
c. providing a unique offset in at least one direction and in at least one size to each of a rest of said EM coil layers in relation to said fixed position of said first EM coil layer.

Example 24. The method according to example 23, further comprising providing a number of EM coils, each having a plurality of EM coil layers to be used on a same orientation.

Example 25. The method according to example 23 or example 24, further comprising providing a geometry of each of said EM coils.

Example 26. The method according to any one of examples 23-25, wherein said identifying a location of traces and spacing on said EM coil is according to said geometry.

Example 27. The method according to any one of examples 23-26, further comprising providing a number of EM coil layers per EM coil.

Example 28. The method according to any one of examples 23-27, further comprising positioning each EM coil layer according to said provided unique offset above or under said first layer.

Example 29. The method according to any one of examples 23-28, further comprising assessing an overall distribution of copper over an entire surface of the EM transmitter to identify possible areas having non-homogenous quantities of copper.

Example 30. The method according to any one of examples 23-29, wherein when areas having non-homogenous quantities of copper have been identified, then the method comprises repeating said providing a unique offset until no areas having non-homogenous quantities of copper have been identified.

Example 31. A method for generating a homogenous or quasi-homogenous distribution of copper over an area of an EM transmitter by providing an inverted layer mask, comprising:
a. providing a number of EM coils to be used on a same orientation;
b. assessing a quantity of copper in the overall areas of the EM transmitter;
c. identifying areas having a higher quantities of copper;
d. setting those higher quantities as a threshold;
e. identifying areas having lower quantities of copper;
f. generating one or more layers comprising traces of copper in the identified areas having lower quantities of copper, thereby generating an inverted layer mask;
g. positioning said one or more generated layers above or below said EM coils, thereby generating an EM transmitter having homogenous or quasi homogeneous copper along the whole surface of the EM transmitter.

Example 32. A method of for generating a homogenous or quasi-homogenous distribution of copper over an area of an EM transmitter, comprising manufacturing PCB of said EM transmitter having a spacing smaller than 3 mil.

Example 33. A tracking system, comprising:
A plurality of EM transmitters, each EM transmitter from said plurality of EM transmitters configured to generate one or more unique EM fields;
wherein each EM transmitter from said plurality of EM transmitters is homogenously translucent under visualization; and wherein superimposed EM transmitters from said plurality of EM transmitters are also homogenously translucent under visualization.

Example 34. An electromagnetic (EM) transmitter, comprising a plurality of EM transmitting coils positioned one on top another; each of said plurality of EM transmitting coils having at least one conductive trace; said EM transmitter defining a flat surface;
wherein said EM transmitter is visually homogeneous or quasi-homogeneous when visualized under means of visualization.

Example 35. The EM transmitter according to example 34, wherein a calculated quantity of conductive trace material in a section along an axis perpendicular to said flat surface of said EM transmitter is homogenous or quasi-homogenous when compared with all other sections in said EM transmitter.

Example 36. The EM transmitter according to example 34 or example 35, wherein said plurality of EM transmitting coils are positioned with an offset in relation to one another in order to generate said homogenous or quasi-homogenous quantity of conductive trace material.

Example 37. The EM transmitter according to any one of examples 34-36, further comprising at least one additional layer; said additional layer comprising one or more regions; said regions having one or more of at least one conductive trace or copper configured for providing material required to achieve said homogenous or quasi-homogenous quantity of conductive material.

Example 38. The EM transmitter according to any one of examples 34-37, wherein one or more EM transmitting coil from said plurality of EM transmitting coils further comprise one or more regions; said regions having one or more of at least one conductive trace or copper configured for providing material required to achieve said homogenous or quasi-homogenous quantity of conductive material.

Example 39. The EM transmitter according to any one of examples 34-38, wherein said plurality of EM transmitting coils are configured to be used with low frequencies and high electrical currents with smaller power dissipation.

Example 40. The EM transmitter according to any one of examples 34-39, wherein:
a. said low frequencies are lower than 1 kHz; and
b. said high electrical currents above 0.3 Ampere or above 1 Ampere.

Example 41. The EM transmitter according to any one of examples 34-40, wherein said plurality of EM transmitting coils are configured to be used with a combination of:
a. low frequencies and high electrical currents; and
b. high frequencies and low electrical currents.

Example 42. The EM transmitter according to any one of examples 34-41, wherein:
a. said low frequencies are lower than 1 kHz;
b. said high electrical currents above 0.3 Ampere or above 1 Ampere;
c. said high frequencies are from 1 kHz to 40 kHz; and
d. said low currents are below 1 Ampere.

Example 43. The EM transmitter according to any one of examples 34-42, wherein said conductive trace material is one or more of copper, silver or any other compatible material.

Example 44. The EM transmitter according to any one of examples 34-43, wherein spacings between parts of said at least one trace are from about 1 mil to about 5 mil.

Example 45. The EM transmitter according to any one of examples 34-44, wherein spacings between parts of said at least one trace are larger than 5 mil.

Example 46. The EM transmitter according to any one of examples 34-45, wherein said at least one conductive trace comprises a weight of from about 0.5 oz to about 20 oz.

Example 47. The EM transmitter according to any one of examples 34-46, each EM transmitting coil from said plurality of EM transmitting coils comprise a geometry of said at least one conductive trace.

Example 48. The EM transmitter according to any one of examples 34-47, wherein said geometry is one or more of square, rectangular, triangular, diagonal, circular, or any other geometrical form.

Example 49. The EM transmitter according to any one of examples 34-48, wherein each EM transmitting coil from said plurality of EM transmitting coils is made of a plurality of sub-transmitting coils.

Example 50. The EM transmitter according to any one of examples 34-49, further comprising an isolating layer between each sub-transmitting coil from said plurality of sub-transmitting coils.

Example 51. The EM transmitter according to any one of examples 34-50, wherein said offset is characterized by a direction and a size.

Example 52. The EM transmitter according to any one of examples 34-51, wherein said direction is one or more of up, down, left and right.

Example 53. The EM transmitter according to any one of examples 34-52, wherein said direction is one or more of in the X axis and/or in the Y axis.

Example 54. The EM transmitter according to any one of examples 34-53, wherein said size is from about 0.01 mm to about 10 mm.

Example 55. The EM transmitter according to any one of examples 34-54, wherein said at least one additional layer is an invert layer or contains copper regions which serve as invert copper to other layers.

Example 56. A tracking system comprising:
a. an EM transmitter according to example 34,
b. a fluoroscope.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1a-b are schematic representations of an exemplary tracking system, according to some embodiments of the invention;

FIG. 1c is a schematic representation of an exemplary flat EM transmitter, according to some embodiments of the invention;

FIGS. 2a-c are schematic representations of exemplary geometries of exemplary EM transmitter coils, according to some embodiments of the invention;

FIG. 3 is a schematic enlarged partial top view of a coil, according to some embodiments of the invention;

FIGS. 4a-e are images of resulting visibility of a PCB transmitter under standard fluoroscopy in three different cases, according to some embodiments of the invention;

FIGS. 5a-b are schematic representations of partial top view and cross-sectional view, respectively, of a multilayered EM coil transmitter having an offset, according to some embodiments of the invention;

FIG. 5c is a schematic cross-section representation of an exemplary multi-layer transmitter, according to some embodiments of the invention;

FIG. 6a is a schematic representation of the concept of an invert layer, according to some embodiments of the invention;

FIGS. 6b-c are schematic representations of exemplary invert layers, according to some embodiments of the invention;

FIGS. 6d-e are schematic representations of exemplary “perfect” invert layers, according to some embodiments of the invention;

FIG. 7 is a flowchart of an exemplary method of generating a homogenous or quasi-homogenous distribution of copper over the area of the EM transmitter by offset positioning, according to some embodiments of the invention;

FIG. 8 is a flowchart of an exemplary method of generating a homogenous or quasi-homogenous distribution of copper over the area of the EM transmitter by providing an inverted layer mask, according to some embodiments of the invention; and

FIG. 9 is a flowchart of an exemplary method of generating a homogenous or quasi-homogenous distribution of copper over the area of the EM transmitter by manufacturing PCB having smaller spacing, according to some embodiments of the invention.

### DESCRIPTION OF SPECIFIC EMBODIMENTS OF THE INVENTION

The present invention, in some embodiments thereof, relates to flat electromagnetic (EM) transmitters and, more particularly, but not exclusively, to flat electromagnetic (EM) transmitters having a negligible interference with other devices.

### Overview

An aspect of some embodiments of the invention relates to EM transmitters that do not interfere a user to read images that include the EM transmitter in the field of view (FOV) of the visualization device. In some embodiments, the EM transmitter comprises one or more EM coils, for example the EM transmitter comprises three or more EM coils. In some embodiments, each coil comprises a PCB including traces having a certain geometry. In some embodiments, the EM transmitter comprises traces with high quantities of copper that allow the EM transmitter to use low frequencies and relatively high electrical currents with smaller power dissipation. In some embodiments, each coil comprises a plurality of layers. In some embodiments, the EM transmitter is a flat EM transmitter having a square or rectangular form. In some embodiments, the EM transmitter comprises an identical or quasi-identical amount of copper at any and/or every section of the EM transmitter, where the area is an area calculated as perpendicular to the surface of the flat EM transmitter—for example, an area when looking at the EM transmitter from above seeing the plurality of coils one on top of another. In some embodiments, each of the plurality of coils are arranged with a calculated offset one form another to provide the identical or quasi-identical amount of copper. In some embodiments, the calculated offset takes under consideration areas in each coil that comprise traces and areas that do not comprise traces to generate an overall homogenous or quasi-homogenous distribution of the copper in the traces over the whole surface of the EM transmitter when observed from above. The terms “homogenous” and “quasi-homogeneous” refer herein after as a subjective view of the EM transmitter under general conditions in a general non-specific fluoroscope as being homogeneous under fluoroscopy imaging (or other means of visualization). In some embodiments, the EM transmitter comprises an additional mask configured to fill gaps in the overall area with copper traces in order to achieve the identical or quasi-identical amount of copper. In some embodiments, the EM transmitter comprises electrically disconnected (floating) coils within a same transmitter coil configured to fill gaps in the overall area with copper traces, similar in pattern to the transmitting coils, in order to achieve the identical or quasi-identical amount of copper. In some embodiments, continuing a winding coil to fill an area of the transmitter with copper has the advantage of maintaining the same copper pattern while filling in gaps with copper. In some embodiments, the identical or quasi-identical amount of copper is achieved by manufacturing a PCB with very small spacing between traces resulting in an overall homogenous or quasi-homogenous distribution of the copper in the traces over the whole surface of the EM transmitter when observed from above. In some embodiments, the EM coils can be positioned in different orientations in order to provide different EM fields. In some embodiments, each EM coil transmits at a unique frequency, which is different from frequencies used in other EM coils within a same EM transmitter.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Introduction to the Problem

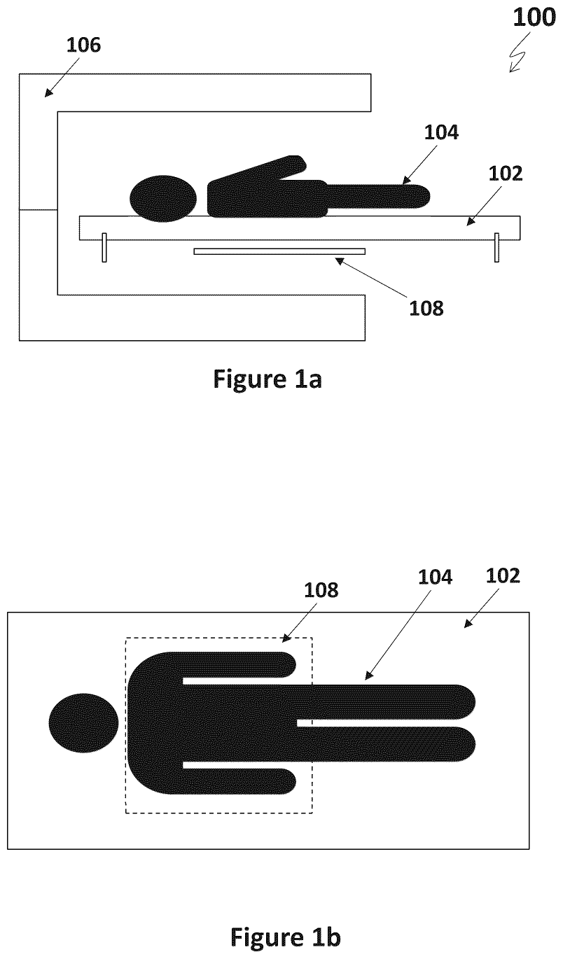

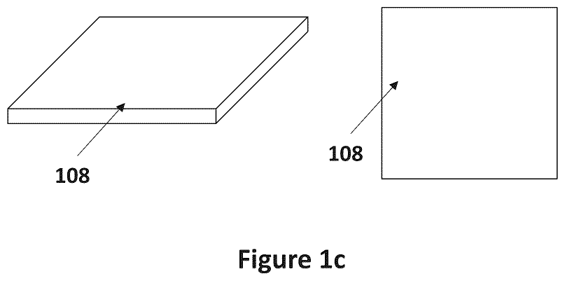

It is known that tracking systems utilize electromagnetic transmitters and receivers to track medical devices within a body of a patient during endoluminal procedures. These systems usually employ a combination of tracking of the medical device by electromagnetic means, while operating simultaneously visual monitoring, for example while employing fluoroscopy. An exemplary system is shown, for example, in FIG. 1a.

Referring now to FIGS. 1a-b, showing an exemplary tracking system, according to some embodiments of the invention. In some embodiments, an exemplary tracking system 100 comprises a bed or a mattress 102 on which a patient 104 is positioned. In some embodiments, the tracking system 100 comprises a fluoroscope 106 and a flat transmitter 108 positioned below the patient 104, for example under the mattress 102. FIG. 1a shows a schematic side view of the tracking system, while FIG. 1b shows a schematic top view of the bed 102, having the patient 104 positioned on the bed 102 and the flat transmitter 108 positioned below the patient 104.

Known tracking systems usually employ high frequencies (for example 1-40 kHz) with relatively low currents (for example, smaller than 1 Ampere) during the tracking procedure due to power and signal-to-noise (SNR) considerations. For example, a coil-based sensor senses an alternating magnetic field according to Faraday's law. For example, an alternating field of intensity I₀ sin(ωt) in the transmitter will generate EMF (Electromotive Force) of intensity correlative to I₀ω·cos(ωt) in the receiver, so a transmitted amplitude of I₀ generates picked up voltage amplitude correlative to I₀ω in the sensing coil, according to Faraday's law of induction. Therefore, it is advantageous to increase ω (the transmitted frequency) in order to increase the pickup in the sensor. Increasing I₀ also increases the pickup in the sensor, but it comes with a greater price. The power dissipation for current I₀ through transmitter coil of resistance R is I₀²R, so it increases with I₀. However, it generally does not increase with ω (for very high ω skin effects may effectively increase R and thus increase the power dissipation). It is therefore advantageous to use high frequencies (for example 1-40 kHz) with rather small currents (for example 0.1 Ampere) to increase the pickup in the sensor while maintaining a rather small power dissipation in the transmitter. In addition, in medical applications, flat electromagnetic (EM) transmitters are used since usually they provide no structural interference with other devices. For example, a flat EM transmitter can be placed on a patient's bed, for example under the patient's mattress, as explained above and shown schematically in FIGS. 1a and 1b. An exemplary flat EM transmitter 108 is shown in FIG. 1c.

Contrary to some known tracking systems, the tracking system of the present invention employs low frequencies with relatively high currents during the procedure. A potential advantage of using low frequencies is that the eddy-currents induced in metals surrounding the system are negligible.

In some embodiments, the system of the invention employs a DC magnetic sensor configured to sense a DC magnetic field, for example by using hall-effect, magnetoresistance, magneto-inductance or other suitable DC magnetic field sensing technique, while, in some embodiments, the system can optionally employ a coil-based sensor that can usually only sense an AC magnetic field due to Faraday's law (i.e., changes in the magnetic field over time). In order to increase the sensor's pickup of the low frequencies, the amplitude of the transmitted field needs to be increased. This is true with a DC magnetometer, where increasing the frequency does not increase the sensor's pickup, and a way to increase the pickup may be to increase the transmitted field's amplitude. In some embodiments, the term “DC magnetometer” refers herein to a sensor which senses DC fields (constant fields) as well as low-mid frequency fields. For example, a magnetic sensor which has a sample rate of 1000 Hz, and it senses magnetic fields with frequencies 0-500 Hz (Nyquist). It should be understood that while its base sensing is for “DC magnetic field”, for the purposes of protection, it is also intended to cover for any “magnetic field”.

To accomplish that, a high-current EM transmitter is used (for example, higher than 0.3 Ampere, higher than 1 Ampere), since the amplitude of the transmitted field is proportional to the electrical transmission current.

In some embodiments, this is the reason why the tracking system of the present invention employs low frequencies with relatively high currents during the procedure.

As mentioned above, in medical application it is advantageous to use a flat EM transmitter, for example, positioned under the mattress of a bed of a patient. In some embodiments, the flat transmitter comprises a printed circuit board (PCB), which includes conductive (for example, copper or silver) traces describing EM transmitting coils for transmitting EM fields. In some embodiments, for these coils to carry high electrical current (for example, higher than 0.3 Ampere or 1 Ampere), it is advantageous for the traces to be wide (for example wider than 1 mm) and/or thick (for example, with copper weight larger than 3 oz). In some embodiments, a potential advantage of increasing the width and/or thickness of copper is that it potentially reduces the resistance of the PCB traces (inversely proportional to increased cross-section area of the traces), which in turn reduces power dissipation of the transmitter for the desired transmission current (according to Watt's law: P=I²R), thus enabling the high electrical current in high efficiency (preventing overheating of the transmitter during operation for a specific desired current I).

It will be appreciated that although the present disclosure refers mostly to copper traces, it should be understood that traces of other suitable metals or conductive materials may be used in some embodiments, with the required respective structural and/or quantitative changes.

However, by increasing the copper (especially in thickness), the PCB traces can become visible in standard fluoroscopy in accordance with Beer-Lambert's law of attenuation (for example, in 80 kVp projections of a standard fluoroscope device such as a C-arm machine). Copper has an approximated mass attenuation coefficient of 0.76 cm²/g under 80 kVp fluoroscopy which can make it highly visible (absorber of X-ray energy) according to Beer-Lambert's law of attenuation. On the other hand, reducing copper width/thickness results in increased power dissipation for the desired transmission current, which can result in increased heating of the transmitter during the procedure. Additionally, manufacturers usually require significantly larger spacing between heavier traces of copper. For example, a 14 mil spacing may be required between copper traces of 5 oz layers, compared to a 3 mil spacing that may be required between copper traces of 1 oz layers. This makes traces of 5 oz even more noticeable on an image generated by standard fluoroscopy, since the spacing between traces is increased. To summarize the issue, wider/thicker traces having larger spacing between the traces generate a very noticeable EM transmitter on an image generated by standard fluoroscopy, but making narrower/thinner tracers is not desirable and currently manufacturers do not or cannot manufacture a PCB with heavy copper and shorter spacing.

Here resides the unique problem to system of the present invention, on one side, the system of the present invention seeks to use lower frequencies and/or DC magnetometers, which necessitate the use the higher currents. Higher currents necessitate increasing the copper in the PCB traces to decrease power dissipation. Increasing the copper in the PCB traces cause the EM transmitter to be seen, for example during the fluoroscopy, which can cause interference during the visualization of the medical device being tracked.

Therefore, an aspect of some embodiments of the invention relates to an EM transmitter having increased copper width/thickness while still maintaining a negligible effect under standard fluoroscopy.

### Exemplary Tracking System

In some embodiments, an exemplary tracking system is as shown for example in FIGS. 1a-1c, with one major difference to known tracking systems, a dedicated EM transmitter. In some embodiments, as mentioned above, the EM transmitter is characterized by having an increased copper width/thickness while still maintaining a negligible visual effect under standard fluoroscopy. In some embodiments, as mentioned above, the EM transmitter is used for electromagnetic tracking and/or navigational procedures. In some embodiments, the “transparency” of the EM transmitter to fluoroscopy allows the use of a fluoroscope during an EM navigational procedures without distorting the resultant images. As used herein, the term “transparency” or “transparent” or “translucency” or “translucent” of the EM transmitter means “negligible effect to fluoroscopy and/or to other devices”. It should be emphasized that the EM transmitter is not actually transparent when a fluoroscope is activated, but rather, as will be further explained below, its presence in the field of view (FOV) of the fluoroscope is negligible to the tracking process.

In some embodiments, the fluoroscope may project anteroposterior (AP) images through the transmitter, such that the transmitter does not add any significant artifacts to the fluoroscopic image. For example, the traces of the transmitter are not noticeable on the image, or are nullified enough so as to not disturb a health professional to read the image. In some embodiments, in a similar manner, the transmitter may be used with a computed tomography (CT) system such as, for example, a cone-beam CT (CBCT), for example to provide three-dimensional (3D) imaging, for example during a procedure, without adding significant artifacts to the 3D imaging.

### Exemplary EM Transmitter

In some embodiments, an exemplary EM transmitter is a flat EM transmitter comprising PCB having multiple EM transmitting coils for transmitting respective different EM fields and different frequencies.

In some embodiments, the EM transmitter comprises an increased copper amount or thickness. In some embodiments, the reason for the increase in the amount of copper can be one or more of:

1. Allowing to carry high electrical currents with smaller power dissipation: In some embodiments, increasing the amount of copper reduces the coil resistances and allows to carry high electrical currents, for example higher than 0.3 Ampere, or higher than 1 Ampere (in amplitude, peak-to-peak or RMS) with smaller power dissipation. In some embodiments, a potential advantage of using high currents is that it allow the use of low-frequency EM tracking systems with smaller power dissipation; and

2. Increasing the number of transmitter EM fields. In some embodiments, for example, instead of 3 fields, the transmitter transmits 6 or more fields using 6 or more transmitting coils to support “single sensor” (5-DOF) EM tracking.

In some embodiments, increasing the copper width (rather than thickness) does not add more interference to a fluoroscopic image, since each X-ray will still traverse through the same amount of copper (for example, through 1 oz of copper). However, with increased trace width, a coil cannot be wound in a PCB so densely, so a PCB coil would have to contain less windings and thus power dissipation will increase. On the other hand, increasing copper thickness (rather than width) does add more interference to a fluoroscopic image, but each coil can contain more windings (since the traces are narrower) and thus power dissipation decreases.

### Exemplary Geometries of EM Transmitter Coils

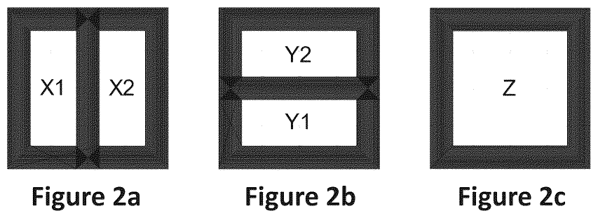

Referring now to FIGS. 2a-c, showing schematic representation of exemplary geometries of exemplary EM transmitter coils, according to some embodiments of the invention. In some embodiments, irrelevantly of the mechanisms that allow an exemplary EM transmitter to be “transparent” to imaging devices, exemplary EM transmitters comprise coils having a certain geometry. In some embodiments, the flat EM transmitter comprises a PCB, which includes metal traces constituting multiple EM transmitting coils for transmitting respective different EM fields.

In some embodiments, each of the multiple coils included in the EM transmitter is configured to transmit a different EM field. For example, coil 1, schematically shown in FIG. 2a, includes two vertically-aligned sub-coils connected in series: sub-coil X1 wound around a first half of the PCB in a first layer, and sub-coil X2 wound around a second half of the PCB, for example, in a second layer. Similarly, coil 2, schematically shown in FIG. 2b, includes two horizontally-aligned sub-coils connected in series, sub-coil Y1 and sub-coil Y2. Coil 3, schematically shown in FIG. 2c, may be wound around the bounds of the PCB, providing a third EM field. In some embodiments, the coils can have any kind of geometry, which are not disclosed in FIGS. 2a-c, for example more than two sub-coils are included in a certain coils (for example 3, 4 5 6 or more sub-coils), the geometry is not square or rectangular, it can be triangular, diagonal, circular, or characterized by any other geometrical form. In the following explanations, rectangular and square geometries will be used to facilitate the explanations, and should be understood that those geometries do not intent to limit the scope of the invention.

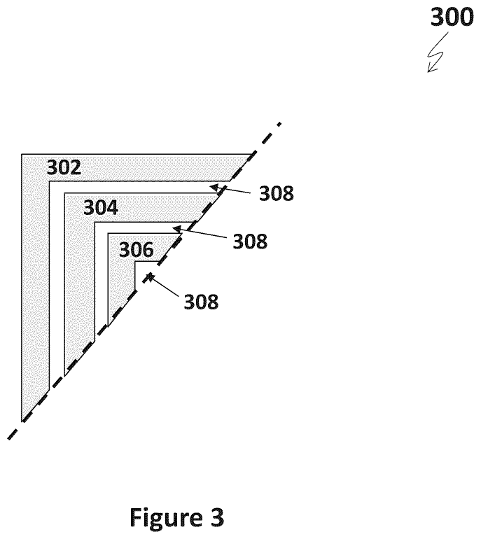

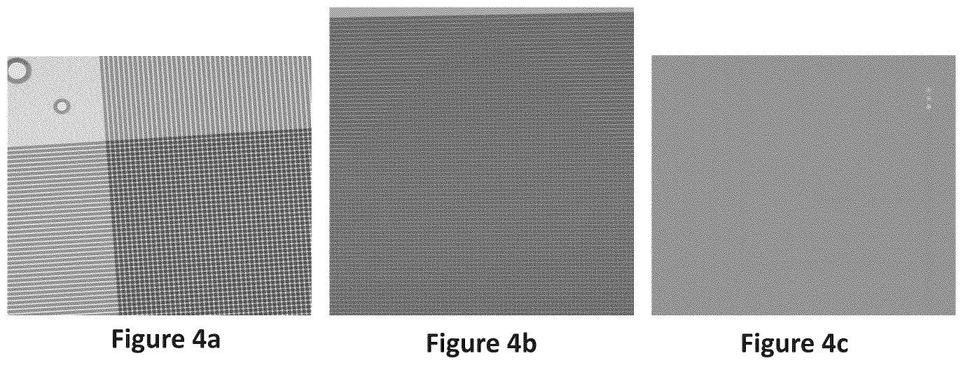

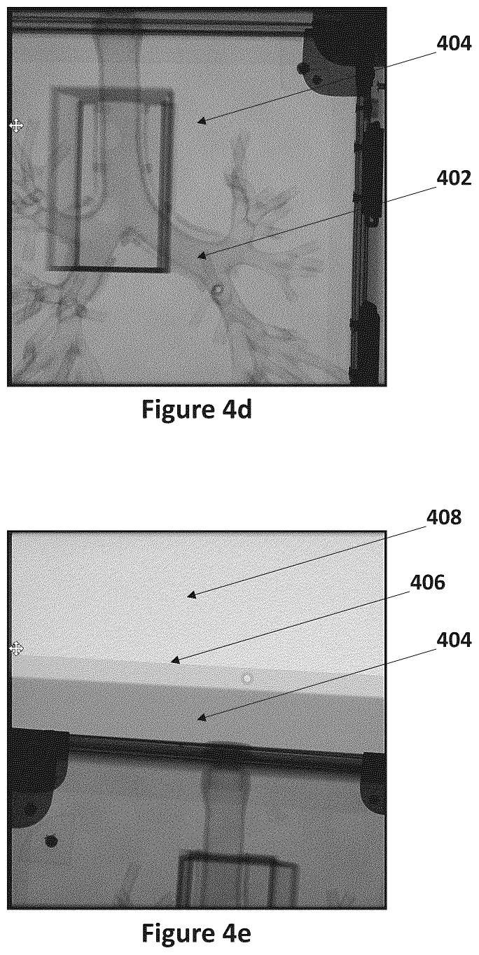

Referring now to FIG. 3, showing a schematic enlarged partial top view of a coil, according to some embodiments of the invention. In some embodiments, an exemplary coil 300 comprises a plurality of traces, for example as shown in FIG. 3, where traces 302, 304 and 306 are shown, which constitute three windings of coil 300. In some embodiments, each of traces 302, 304 and 306 is, for example, 1 mm wide (possibly with a negligible error margin). In some embodiments, the overall height (thickness) of each of traces 302, 304 and 304 may be of Soz PCB layer which amounts to 175 um (possibly with a negligible error margin). In some embodiments, an exemplary coil 300 comprises spacing 308 between the traces. In general, manufacturers usually require about 14 mil spacing between traces of Soz layers. In some embodiments, traces of Soz PCB layer are highly visible in standard fluoroscopy, especially when separated by 14 mil spaces, for example as shown in FIG. 4a (see below).

### Exemplary Multi-Layer Coil

In some embodiments, exemplary coils, having either geometry, comprise a single coil layer. In some embodiments, each exemplary coil, having either geometry, comprise a plurality of coil layers (referred herein as multi-layer coil). In some embodiments, in a certain geometry-type coil, each layer comprises conductive traces optionally having the same geometry. In some embodiments, optionally, multi-layer coils comprise adjacent coils having different geometries. In some embodiments, in this case, the complete coil comprises a unique known geometry and the sub layers are arranged such that the result is fluoroscopy-“transparent” (see below).

In some embodiments, for example, instead of using a single Soz copper coil layer with, for example, 1 mm-wide traces and 14 mil spacing, a 5 oz copper layer is split into 5 aligned layers of 1 oz each, or into 10 aligned layers of 0.5 oz each, with, for example, 1 mm-wide traces and 3 mil spacing. In some embodiments, a potential advantage of splitting a coil into layers is that it potentially reduces spacing between traces, and, as mentioned above, reducing the spacing increases the level of “transparency” of the EM transmitter by making the separation between traces less visible in fluoroscopy. Reducing the spacing also allows to include more windings of an EM coil on a single layer, since the coil can be wrapped more densely. Including more windings increases the coil's inductance and improves power dissipation.

In some embodiments, since the traces between the multiple layers are aligned, the EM transmitting coils are still visible in fluoroscopy due to high effective copper weight (since, according to Beer-Lambert's law of attenuation, the final attenuation is a product of all differential attenuations of a single X ray in space, which traverses through the total amount of copper). In some embodiments, this problem is solved following the solution as explained below.

In some embodiments, the resulting coils (each may consist of multiple PCB layers connected in parallel) may have similar inductance (possibly with a negligible error margin) relative to a single coil which is made of a single (thick) PCB layer. In some embodiments, this may be achieved because each coil comprises multiple parallel coils of the same geometry which are almost perfectly aligned (see below). In some embodiments, the inductance of parallel-connected aligned coils is relatively similar to the inductance of a single coil, due to full mutual inductance between the aligned coils. In addition, parallel-connected aligned coils may introduce mutual capacitance between the coils. However, this capacitance is negligible, especially in the case of a low-frequency EM transmitter.

### Exemplary Principle of the Solution

In some embodiments, in order to overcome the problems generated by the increased amount in copper, either in a single EM transmitting coil or in a multi-layer transmitting coil, the inventors have developed several potential solutions, all which follows a same principle: an EM transmitting coil will have a similar amount of copper at every point or sub-area over the overall area of the transmitter when observed from above (or from some limited range of angles around top view, such as a 45 degrees range).

Referring now to FIGS. 4a-e, showing images of resulting visibility of a PCB transmitter under standard fluoroscopy in three different cases, according to some embodiments of the invention.

FIG. 4a shows the resulting visibility of a transmitter with a single 5 oz layer per coil and 14 mil spaces between traces. It can be seen that the traces are very visible and may interfere with the usability of a fluoroscopic image, e.g. in a manner that may impair a professional's ability to obtain information from the image.

FIG. 4b shows the resulting visibility of a transmitter with five 1 oz aligned layers per coil and 3 mil spaces between traces. It can be seen that the visibility of the traces is reduced, but they are still very visible and may interfere with the usability of the fluoroscopic image.

FIG. 4c shows the resulting visibility of a transmitter with five 1 oz layers per coil, 3 mil spaces between traces. It can be seen that the traces are barely visible, and that this transmitter in this case is substantially fluoroscopy-“transparent” or “translucent”. FIG. 4c shows a principle of an exemplary solution, which is having a substantially similar amount of copper at every point over the overall area of the transmitter when observed from above, or from some angle range from above, for example, from 45 degrees angle range tilt from top view, thereby generating a homogeneous or quasi-homogeneous copper surface that will not interfere with the usability of a fluoroscopic image.

FIGS. 4d-e show fluoroscopic images demonstrating the visual homogeneity of an exemplary transmitter, according to some embodiments of the invention. FIG. 4d shows the clear visualization of the plastic lung model 402 over the homogenous background 404 of the transmitter, while FIG. 4e shows the border 406 of the transmitter, in which it can clearly be seen the difference between an area 404 where the transmitter is located over an area 408 where there is no transmitter and how homogenous the transmitter looks like under fluoroscopy.

In the following paragraphs, possible solutions to achieve a transmitter that is substantially fluoroscopy-“transparent” will be provided.

### Exemplary Solutions

Reducing the Spacing in the PCB In some embodiments, an exemplary method of generating a homogeneous or quasi-homogeneous copper surface that will not interfere with the usability of a fluoroscopic image is by reducing the spacing between traces. For example, by producing 1 oz/0.5 oz layers with very small spacing (1 mil, 2 mil, 3 mil), then the resulting PCB will be effectively “transparent”. In some embodiments, the quality of the fluoroscope also influences the image. For example, the better the fluoroscope resolution is, the smaller this spacing should be. In some embodiments, with standard systems, 3 mil spacing may suffice, as long as the number of such 3 mil spacings that align with each other between different layers is small. In some embodiments, 1 mil spacing may be sufficient even with alignment.
Overlapping EM Transmitting Coils with an Offset

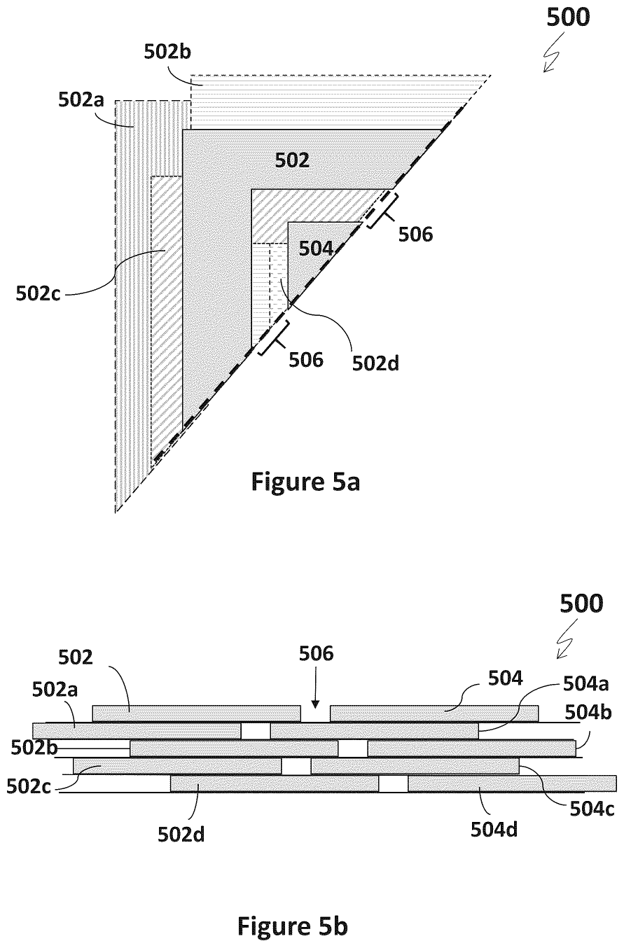

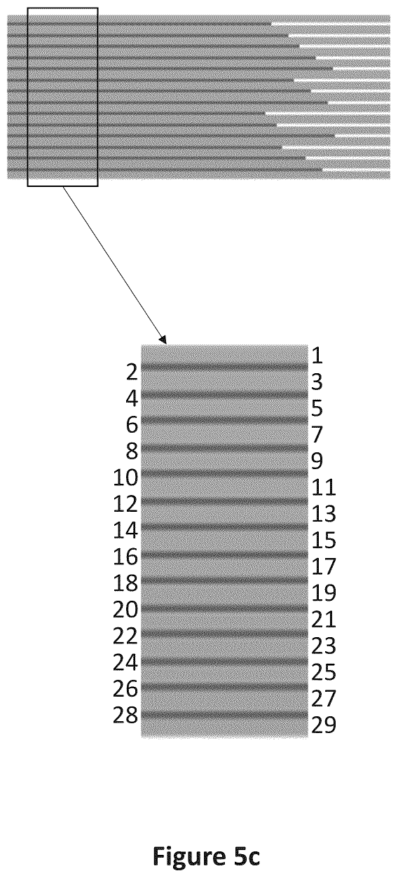

Referring now to FIGS. 5a-b, showing schematic representations of partial top view and cross-sectional view, respectively, of a multilayered EM coil transmitter having an offset, according to some embodiments of the invention.

As mentioned in relation to FIG. 3, manufacturers usually require about 14 mil spacing between traces of 5 oz layers, and traces of 5 oz PCB layer are highly visible in standard fluoroscopy, especially when separated by 14 mil spaces. In some embodiments, as mentioned above, each 5 oz layer is split into multiple layers of 1 oz each, or of 0.5 oz each, with, for example, 1 mm-wide traces and 3 mil spacing, where each of the coil layers in each EM transmitting coil are aligned with an offset relative to the other coil layers. Thus, for example, the offset traces cover each other's spacing between traces, and the tracing becomes much less noticeable in the fluoroscopic image. In some embodiments, the offset is characterized by a direction and a size. In some embodiments, the direction is one or more of up, down, left and right. In some embodiments, the direction is defined as movement in the X axis and/or in the Y axis. In some embodiments, the size is from about 0.01 mm to about 10 mm.

Referring now to FIG. 5a, showing a schematic representation of traces of different EM coils being over-layered with an offset, according to some embodiments of the invention. In some embodiments, multilayered coil 500 may include multiple coil windings, including windings 502 and 504, with a spacing 506 between them. In some embodiments, winding 502, representing any winding of coil 500, may include multiple layers, for example five layers 502, 502a, 502b, 502c and 502d. Similarly, for example, winding 504 includes multiple layers 504 and 504a to 504d (not shown). In some embodiments, each layer may be of, for example, loz or 0.5 oz (possibly with a negligible error margin). In some embodiments, spacing 506 may be of 3 mil (possibly with a negligible error margin). In some embodiments, optionally, copper layers are separated by thin insulation layers (see below FIG. 5c) to prevent short circuit between different layers, as in standard PCB manufacturing processes.

In some embodiments, each layer is positioned with a unique offset in relation to the others, for example as explained above, in a different direction and/or of a different magnitude (size), relative to the other layers. In some embodiments, the offsets are calculated for all of the coils and layers of transmitter 108, so that x-rays, which traverse transmitter 108, pass through a relatively similar amount of copper (or any other suitable tracing metal) in various locations across transmitter 108 and, in some embodiments, in various angles of the x-rays (for example, depending on the position and orientation of a C-arm machine). In some embodiments, for example, in the case of three EM coils, each having five layers, the PCB may have 15 layers, each having a different offset. In some embodiments, the offsets are used to blend the layers in a mixture such that the resulting PCB is effectively “transparent” under standard fluoroscopy. For example, in case the traces are 1 mm wide with 3 mil spacing between the traces, the offset magnitudes may be smaller than 1.08 mm (which is the sum of trace width plus trace spacing). In some embodiments, the reason for this is that the wound coils can be 1.08 mm periodic, so that offsetting by more than 1.08 mm cycles back to offsetting by the modulus of the offset by the 1.08 mm period.

In some embodiments, the ideal offsets are found in a fluoroscopic simulation according to Beer-Lambert's law of attenuation, e.g. based on the assumption that there is an exponential relationship between the amount of copper traversed by an x-ray and the radiation intensity the x-ray loses in its path, according to Beer-Lambert's law of attenuation.

In some embodiments, the calculation of the required offset is done manually by a user, which can freely “move” the plurality of layers, for example, in a simulation program for designing EM transmitters.

In some embodiments, the calculation of the required offset is done automatically by a dedicated software, having instructions to generate n possible combinations of movements of the layers until reaching a homogenous or quasi-homogenous distribution of copper over the area of the EM transmitter.

In some embodiments, the calculation of the required offset is done automatically by a dedicated software, having instructions to converge to a homogenous or quasi-homogenous distribution of copper over the area of the EM transmitter by using non-linear optimization methods, such as Gradient Descent, Levenberg Marquardt, or any other suitable method. In some embodiments, the software may begin at some initial guess (for example, a random guess or a random value of offset) and converge locally or globally using non-linear optimization methods to minimize an energy function which describes the non-uniformity of a simulated fluoroscopic image, based on the current parametrized coil layer offsets.

Referring now to FIG. 5c, showing a schematic cross-section representation of an exemplary multi-layer transmitter, according to some embodiments of the invention. FIG. 5c shows an exemplary multi-layer transmitter (layers not in scale) where layers of copper are separated by layers of insulation material. For example, the odd numbers (1, 3, 5, 7, etc.) are the insulation layers, while the even numbers (2, 4, 6, 8, etc.) are the copper traces. Exemplary sizes are for example: insulation layer about 0.09 mm, copper layer (1 oz) about 0.035 mm.

### Providing an Invert Layer

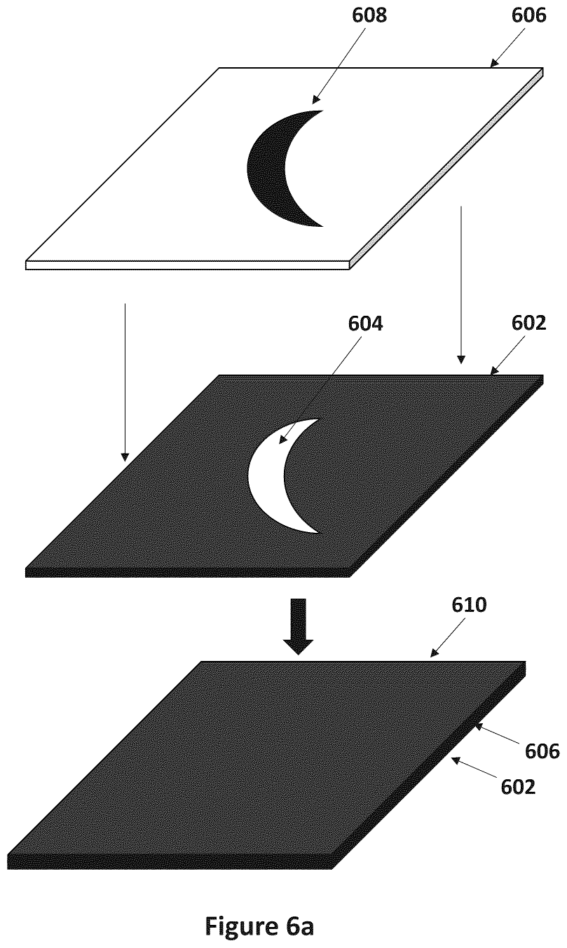

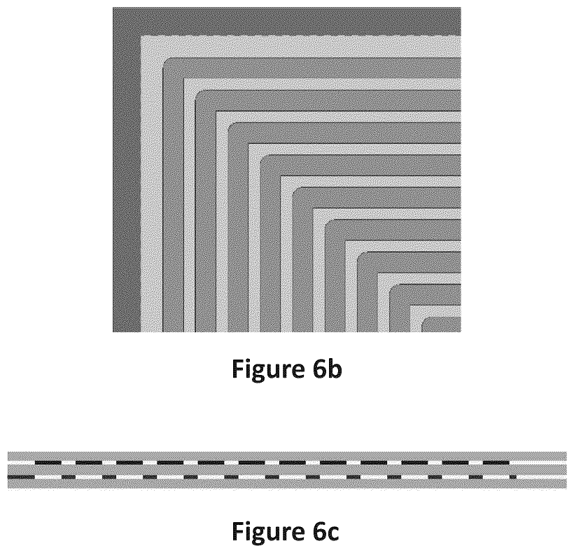

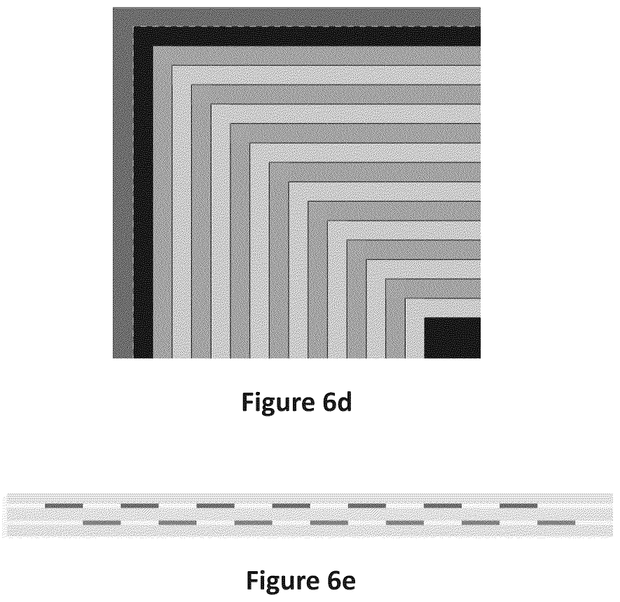

Referring now to FIG. 6a, showing a schematic representation of the concept of an invert layer, according to some embodiments of the invention. In some embodiments, another option to nullify visibility of PCB traces of a flat EM transmitter in a fluoroscopic image comprises providing each PCB coil layer with an invert layer, for example, a PCB layer that includes copper in all areas where the coil layer has no copper, thus making the traces invisible in the resulting fluoroscopic image. This concept is schematically shown in FIG. 6a, where a PCB 602 is covered by copper, but at a location in the center 604. The invert layer 606, comprises a copper trace 608 at the location where copper is missing 604 in the PCB 602. Once mounted, the two layers will provide a uniform layer of copper 610. In some embodiments, this can be achieved, for example, by utilizing each PCB inner core (rigid base material laminated with copper on one or two sides) by etching the transmitting coil on one side (for example, on top side) and the accompanying invert layer on the other side (for example, on bottom side). In this way each invert layer is always closest to its positive layer to achieve maximum transparency of the resulting fluoroscopic image.

In some embodiments, an invert layer may contain copper which is missing in its coil layer counterpart alone. In some embodiments, an invert layer may contain copper which is missing in more than just a single layer of the PCB. For example, multiple coil layers may contain spacings and other parts with missing copper. In some embodiments, a single invert layer may contain the total copper of those multiple coil layers in a single invert layer.

Referring now to FIGS. 6b-c, showing schematic representations of exemplary invert layers, according to some embodiments of the invention. FIG. 6b shows a schematic upper partial view of a transmitter showing two layers, best seen in FIG. 6c in the cross section view, and the complementary coverage of copper, practically providing an overall homogenous or quasi-homogenous distribution of copper over the transmitter.

Referring now to FIGS. 6d-e, showing schematic representations of exemplary “perfect” invert layers, according to some embodiments of the invention. In some embodiments, adding an invert layer may provide fluoroscopic-“translucency”, but adds copper to the total copper amount of the PCB. In some embodiments, an invert layer may be used as a transmitting coil layer—or two transmitting coil layers are designed to be the inverted layer to one another. FIG. 6d shows a schematic upper partial view of a transmitter showing two layers, best seen in FIG. 6e in the cross section view, and the complementary coverage of copper, practically providing an overall homogenous or quasi-homogenous distribution of copper over the transmitter. For example, a PCB coil can comprise 1 mm trace with 1 mm spacing between traces. In some embodiments, as long as the coil winds regularly, it may be possible to “offset it” by exactly 1 mm such that copper and spacing will switch positions. In this embodiment, one layer may contain a coil, and another layer may contain a 1 mm shifted coil, such that each coil trace covers the other layer's spacing in between traces. In this embodiment, the two layers serve as invert layers, but they both also serve as transmitting coil layers, so that all copper is a transmitting copper, instead of placing dead isolated (floating) copper just for balancing the visual non-uniformity of the fluoroscopic image. In some embodiments, those complementary coils can connect in parallel or in series and be parts of a single transmitting coil, or they can belong to separate transmitting coils.

### Low-High-Frequency Hybrid Transmitter

As mentioned above, a low-frequency transmitter requires relatively high electrical current to increase the pickup in the sensor. However, the same transmitter can be used in order to transmit both low-frequency fields as well as high-frequency fields in parallel. In some embodiments, this is achieved, for example, by driving low-frequency high-current sine waves into the transmitting coils, superimposed with high-frequency low-current sine waves into the same coils. For example, a low-frequency transmitter which comprises 3 transmitting coils may transmit the following currents through its 3 coils: I₀ sin(1ω₀t), I₀ sin(2ω₀t), I₀ sin(3ω₀t), where ω₀=2πf₀ and f₀ is some base low transmission frequency (for example, f₀=30 Hz). Sometimes ω and f are both referred to as “frequencies” although ω may also be referred to as angular speed. In a low-high-frequency transmitter, high frequencies may be added to each of the transmitter coils, for example to transmit the following currents through its 3 coils:

$${I}_{0} sin ( 1 {ω}_{0} t ) + {I}_{1} sin ( 100 \cdot 1 {ω}_{0} t )$$

$${I}_{0} sin ( 2 {ω}_{0} t ) + {I}_{1} sin ( 100 \cdot 2 {ω}_{0} t )$$

$${I}_{0} sin ( 3 {ω}_{0} t ) + {I}_{1} sin ( 100 \cdot 3 {ω}_{0} t )$$

Where for example I₀=1 Ampere, I₁=0.1 Ampere, f₀=30 Hz such that the transmitted low frequencies are: 30 Hz, 60 Hz, 90 Hz and the superimposed transmitted high frequencies are: 3 kHz, 6 kHz, 9 kHz.

The large amount of copper described herein supports low-frequency high-current signals by providing reduced resistance of each transmitting coil, let alone it supports high-frequency low-current signals, so that the superimposed high-frequency signals are easily transmitted together with the low frequency.

In some embodiments, the low-high-frequency transmitter can be used to support both low-frequency EM tracking system (for example, which makes use of DC magnetometers) as well as high-frequency “traditional” coil-based EM tracking system. The same low-high-frequency transmitter can track both kinds of sensors in the same procedure: low-frequency sensors (for example, which are based on DC magnetometers) as well as high-frequency sensors (for example, which are based on EM coils and the Faraday law of induction, with high frequencies for example from 1 kHz to 40 kHz).

In some embodiments, the receivers can be independent, each using its own frequency range, such that the low-frequency receivers will not be interfered by the high-frequency transmitted EM fields, because they only listen on specific low frequencies and ignore high frequencies, and the high-frequency receivers will not be interfered by the low-frequency transmitted EM fields, because they only listen on specific high frequencies and ignore low frequencies.

In some embodiments, supporting both kinds of EM sensors can be beneficial in adding features to existing coil-based EM systems, in which existing devices which are coil based are already used, for example in a clinical scenario. The hybrid low-high-frequency transmitter can then support the existing devices, while adding features from new devices which are low-frequency based.

Using low-frequency EM tracking is beneficial in providing cost-effective shape and position tracking, as well as reducing EM metal distortion effects. Adding those abilities to existing high-frequency tracking platforms, rather than completely replacing those platforms with low-frequency ones, can provide back compatibility with existing products, smooth transition to new low-frequency based devices, as well as just supporting both low-frequency and high-frequency EM tracking techniques, such that each tracked device can then use the technology which best fits its needs, under the same tracking platform.

A low-high-frequency transmitter can provide a single platform which supports both low-frequency and high-frequency EM tracking for various uses.

### Exemplary Use of Multiple Transmitters

In some embodiments, multiple fluoroscopy-transparent transmitters may be placed in a clinical setting at different positions and/or orientations to generate an increased number of different transmitted EM fields (for example, more than 3 fields). In some embodiments, the transmitters are synchronized and configured to use different frequencies. For example, two 3-coil transmitters are placed perpendicularly, parallel, one on top of the other, one next to the other or at any other suitable configuration such that they both transmit a total number of 6 different EM fields at 6 respective different frequencies. For example, in one embodiment, two 3-coil transmitters lie one on top of the other and are rotated by 45 degrees. Since each of the transmitters is “transparent” to fluoroscopy, the combined configuration is also “transparent”. In some embodiments, a potential advantage of doing this is that it potentially allows to use multiple “simple” transmitters (e.g., 3-coil transmitter) to generate more EM fields (for example, 6 fields or more) for example to support 5-DOF sensor tracking. In some embodiments, the mechanical configuration of the transmitters is calibrated prior to procedure using EM calibration methods.

### Exemplary Methods

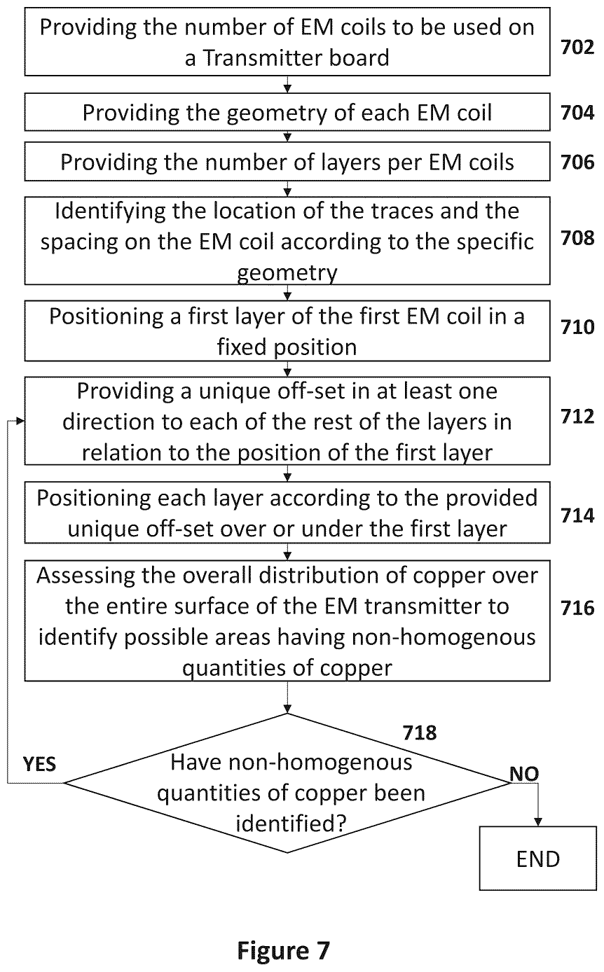

Referring now to FIG. 7, showing a flowchart of an exemplary method of generating a homogenous or quasi-homogenous distribution of copper over the area of the EM transmitter by offset positioning, according to some embodiments of the invention.

In some embodiments, the method comprises one or more of the following actions:

Providing the number of EM coils to be used on a Transmitter board 702.

Providing the geometry of each EM coil 704.

Providing the number of layers per EM coils 706.

Identifying the location of the traces and the spacing on the EM coil according to the specific geometry 708.

Positioning a first layer of the first EM coil in a fixed position. The fix position being used as reference for the offset for the rest of the layers 710.

Providing a unique offset in at least one direction and in at least one size to each of the rest of the layers in relation to the position of the first layer 712.

Positioning each layer according to the provided unique offset over or under the first layer 714.

Assessing the overall distribution of copper over the entire surface of the EM transmitter to identify possible areas having non-homogenous quantities of copper 716.

When areas having non-homogenous quantities of copper have been identified—Repeating actions 712, 714 and 716—until no areas having non-homogenous quantities of copper have been identified 718.

When no areas having non-homogenous quantities of copper have been identified—then the method ends.

In some embodiments, this method is performed for any and all EM transmitters and/or EM transmitter coils needed to be arranged. For example, additional EM transmitters being superimposed with the first EM transmitters and/or additional EM transmitters positioned at a different angle form the first ones.

In some embodiments, assessing the overall distribution of copper over the entire surface can be done by calculating an energy function which measures the non-uniformity of copper, for example, by generating a simulated fluoroscopic image of the current offset configuration (according to Beer-Lambert's law) and computing the simulated image 2D standard deviation.

In some embodiments, reducing the overall non-uniformity of copper distribution can be done by striving to minimize the above energy function, thus bringing the 2D standard deviation of the simulated fluoroscopic image closer to zero.

In some embodiments, the actions above are performed manually by a user. In some embodiments, the actions above are performed automatically by a dedicated software.

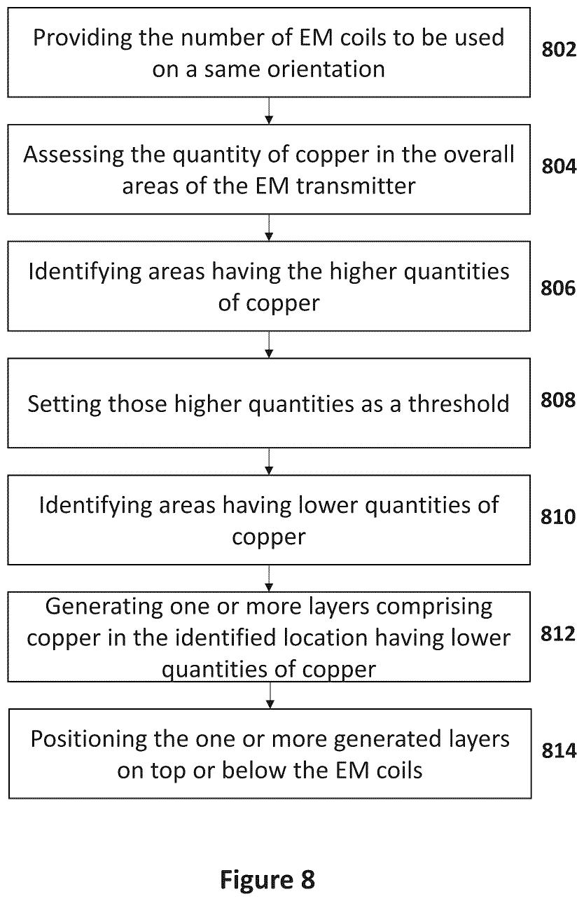

Referring now to FIG. 8, showing a flowchart of an exemplary method of generating a homogenous or quasi-homogenous distribution of copper over the area of the EM transmitter by providing an inverted layer mask, according to some embodiments of the invention.

In some embodiments, the method comprises one or more of the following actions:

Providing the number of EM coils to be used on a same orientation 802.

Assessing the quantity of copper in the overall areas of the EM transmitter 804.

Identifying areas having the higher quantities of copper 806.

Setting those higher quantities as a threshold 808.

Identifying areas having lower quantities of copper 810.

Generating one or more layers comprising traces of copper in the identified location having lower quantities of copper 812, thereby generating an inverted layer mask.

Positioning the one or more generated layers on top or below the EM coils 814, thereby generating an EM transmitter having homogenous or quasi homogeneous copper along the whole surface of the EM transmitter when looked form above.

In some embodiments, the actions above are performed manually by a user. In some embodiments, the actions above are performed automatically by a dedicated software.

In some embodiments, the method of generating a homogenous or quasi-homogenous distribution of copper over the area of the EM transmitter by providing an inverted layer mask is done in addition or instead of the method of generating a homogenous or quasi-homogenous distribution of copper over the area of the EM transmitter by offset positioning.

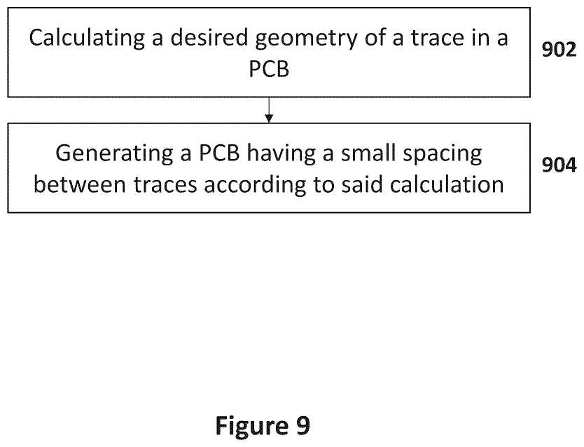

Referring now to FIG. 9, showing a flowchart of an exemplary method of generating a homogenous or quasi-homogenous distribution of copper over the area of the EM transmitter by manufacturing PCB having smaller spacing, according to some embodiments of the invention.

In some embodiments, a method of generating a homogenous or quasi-homogenous distribution of copper over the area of the EM transmitter is by calculating a desired geometry of a trace in a PCB 902 and then manufacturing the PCB having smaller spacing 902 according to said calculation. In some embodiments, the smaller the spacing, the more negligible effect will have the spacing on the resulting fluoroscopic image. In some embodiments, the spacing is below 3 mil. In some embodiments, the spacing is from about 3 mil to about 1 mil.

It is expected that during the life of a patent maturing from this application many relevant PCB manufacturing technologies will be developed; the scope of the present invention is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An electromagnetic (EM) transmitter, comprising a plurality of EM transmitting coils positioned one on top another; each of said plurality of EM transmitting coils having at least one conductive trace; said EM transmitter defining a flat surface;
wherein a calculated quantity of conductive trace material in a section along an axis perpendicular to said flat surface of said EM transmitter is homogenous or quasi-homogenous when compared with all other sections in said EM transmitter.

2. The EM transmitter according to claim 1, wherein said plurality of EM transmitting coils are positioned with an offset in relation to one another in order to generate said homogenous or quasi-homogenous quantity of conductive trace material.

3. The EM transmitter according to claim 1, further comprising at least one additional layer; said additional layer comprising one or more regions; said regions having one or more of at least one conductive trace or copper configured for providing material required to achieve said homogenous or quasi-homogenous quantity of conductive material.

4. The EM transmitter according to claim 1, wherein one or more EM transmitting coil from said plurality of EM transmitting coils further comprise one or more regions; said regions having one or more of at least one conductive trace or copper configured for providing material required to achieve said homogenous or quasi-homogenous quantity of conductive material.

5. The EM transmitter according to claim 1, wherein said plurality of EM transmitting coils are configured to be used with low frequencies and high electrical currents with smaller power dissipation.

6. The EM transmitter according to claim 5, wherein:
a. said low frequencies are lower than 1 kHz; and
b. said high electrical currents above 0.3 Ampere or above 1 Ampere.

7. The EM transmitter according to claim 1, wherein said plurality of EM transmitting coils are configured to be used with a combination of:
a. low frequencies and high electrical currents; and
b. high frequencies and low electrical currents.

8. The EM transmitter according to claim 7, wherein:
a. said low frequencies are lower than 1 kHz;
b. said high electrical currents above 0.3 Ampere or above 1 Ampere;
c. said high frequencies are from 1 kHz to 40 kHz; and
d. said low currents are below 1 Ampere.

9. The EM transmitter according to claim 1, wherein said conductive trace material is one or more of copper, silver or any other compatible material.

10. The EM transmitter according to claim 1, wherein spacings between parts of said at least one trace are from about 1 mil to about 5 mil or larger than 5 mil.

11. (canceled)

12. The EM transmitter according to claim 1, wherein said at least one conductive trace comprises a weight of from about 0.5 oz to about 20 oz.

13. The EM transmitter according to claim 1, each EM transmitting coil from said plurality of EM transmitting coils comprise a geometry of said at least one conductive trace; and wherein said geometry is one or more of square, rectangular, triangular, diagonal, circular, or any other geometrical form.

14. (canceled)

15. The EM transmitter according to claim 1, wherein each EM transmitting coil from said plurality of EM transmitting coils is made of a plurality of sub-transmitting coils; and further comprising an isolating layer between each sub-transmitting coil from said plurality of sub-transmitting coils.

16. (canceled)

17. The EM transmitter according to claim 2, wherein said offset is characterized by a direction and a size; and wherein at least one of the following is true:
a. said direction is one or more of up, down, left and right;
b. said direction is one or more of in the X axis and/or in the Y axis; and
c. said size is from about 0.01 mm to about 10 mm.

18-20. (canceled)

21. The EM transmitter according to claim 3, wherein said at least one additional layer is an invert layer or contains copper regions which serve as invert copper to other layers.

22. (canceled)

23. A method for generating a homogenous or quasi-homogenous distribution of copper over an area of an EM transmitter by offset positioning, comprising:
a. identifying a location of traces and spacing on a plurality of EM coil layers;
b. positioning a first EM coil layer in a fixed position thereby generating a reference layer for said offset;
c. providing a unique offset in at least one direction and in at least one size to each of a rest of said EM coil layers in relation to said fixed position of said first EM coil layer.

24. The method according to claim 23, further comprising providing a number of EM coils, each having a plurality of EM coil layers to be used on a same orientation.

25. The method according to claim 24, further comprising providing a geometry of each of said EM coils; and wherein said identifying a location of traces and spacing on said EM coil is according to said geometry.

26. (canceled)

27. The method according to claim 23, further comprising providing a number of EM coil layers per EM coil.

28. The method according to claim 23, further comprising positioning each EM coil layer according to said provided unique offset above or under said first layer.

29. The method according to claim 23, further comprising assessing an overall distribution of copper over an entire surface of the EM transmitter to identify possible areas having non-homogenous quantities of copper; and
wherein when areas having non-homogenous quantities of copper have been identified, then the method comprises repeating said providing a unique offset until no areas having non-homogenous quantities of copper have been identified.

30-56. (canceled)
